(19) United States
(12) Patent Application Publication
(10) Pub. No.: US 2025/0213454 A1
(43) Pub. Date: Jul. 3, 2025

(54) COSMETIC COMPOSITION WITH IMPROVED SKIN ABSORPTION

(71) Applicant: LG H&H CO., LTD., Seoul (KR)

(72) Inventors: Na Eun YOOK, Seoul (KR); Hyun Min HWANG, Seoul (KR)

(73) Assignee: LG H&H CO., LTD., Seoul (KR)

(21) Appl. No.: 18/850,395

(22) PCT Filed: Mar. 24, 2023
(86) PCT No.: PCT/KR2023/003939

§ 371 Date: Sep. 24, 2024

(30) Foreign Application Priority Data
Mar. 24, 2022 (KR) 10-2022-0036811

(51) Int. Cl. A61K 8/67; A61K 8/365; A61Q 19/00

(52) U.S. Cl. CPC A61K 8/675; A61K 8/365; A61Q 19/00

(57) ABSTRACT

Provided are a cosmetic composition with improved skin absorption and a method of using the composition which includes niacinamide and citric acid together and thus does not allow niacinamide to remain on the skin even when the solvent and other additives evaporate or are absorbed after application of the cosmetic composition to the skin, and solve usability issues caused by residue or white spots resulting from the remaining niacinamide.

### TECHNICAL FIELD

The present invention relates to a cosmetic composition with improved skin absorption.

### BACKGROUND ART

Niacinamide (or nicotinamide), a type of niacin and a member of the vitamin B group, is a precursor of the intracellular antioxidant NADPH, and is converted into NADPH in the body and closely involved in cellular antioxidant functions, bioenergy production processes, and various physiological functions of various enzymes.

Previously, several studies on niacinamide have been conducted, and many reports have been made about its various effects. Particularly, the lightening effect of niacinamide (EP 0396422 A1), the moisturizing effect by improving the skin barrier (BR. J. Dermatol. 2000, 143(3), 524-31), and the anti-inflammatory and inflammatory acne improvement effect (Int J Dermato. 1995, 34: 434-437), and an anti-aging effect that improves wrinkles and elasticity (Dermatol Surg. 2005, 31: 860-865) have been reported.

As the cosmetics industry develops, the development of highly functional cosmetics continues to take place throughout the cosmetics industry. Recently, as the number of consumers demanding specific effects such as brightening, wrinkle improvement, elasticity, and pore improvement has increased, the value of functional cosmetics with components having enhanced efficacy is increasing in the cosmetics industry.

Particularly, niacinamide, a water-soluble vitamin B3, is widely used in cosmetics because it has various effects on the skin as described above, and recently, many cosmetics containing niacinamide in a high content of 5% or more to enhance the efficacy of niacinamide have been released.

However, when niacinamide was simply mixed at a high content and applied to cosmetic preparations, the perceived effect was not that great compared to the content. This is because when a cosmetic preparation is applied to the skin, the transdermal absorption rate of niacinamide, a water-soluble material, is significantly slower than the evaporation or absorption rate of the solvent, so that it is not absorbed and remains on the skin. Therefore, when a high content of niacinamide is applied, not all of the content penetrates the skin and some remains white on the skin, so not only is the efficacy not sufficient compared to the applied content, but white marks on the skin reduces aesthetics and gives a bad feeling of use due to the foreign body sensation.

Under the above-described background, the present inventors used niacinamide and citric acid together, and when a cosmetic composition containing a high content of niacinamide is applied to the skin and the formulation evaporates, it was confirmed that the niacinamide did not leave a white residue and that skin penetration was improved.

### DISCLOSURE

### Technical Problem

The present invention is directed to providing a cosmetic composition which, when applied to the skin and the formulation evaporates, does not leave white residues even when comprising high contents of niacinamide, and which has improved skin penetration.

### Technical Solution

The present invention provides a cosmetic composition including: niacinamide; and citric acid,

wherein the content of the niacinamide is 5% by weight or more based on the total weight of the cosmetic composition, and

the molar ratio of the niacinamide and the citric acid is 8:1 to 1:4.5.

In addition, the present invention provides a method of increasing the skin absorption and/or skin penetration of niacinamide using a cosmetic composition including: niacinamide; and citric acid,

wherein the content of the niacinamide is 5% by weight or more based on the total weight of the cosmetic composition, and

the molar ratio of the niacinamide and the citric acid is 8:1 to 1:4.5.

In addition, the present invention provides a method of reducing the amount of residual niacinamide on the skin using a cosmetic composition including: niacinamide; and citric acid,

wherein the content of the niacinamide is 5% by weight or more based on the total weight of the cosmetic composition, and

the molar ratio of the niacinamide and the citric acid is 8:1 to 1:4.5.

The present invention provides the use of a cosmetic composition for use in the manufacture of cosmetics for increasing the skin absorption and/or skin penetration of niacinamide, the cosmetic composition including: niacinamide; and citric acid,

wherein the content of the niacinamide is 5% by weight or more based on the total weight of the cosmetic composition, and

the molar ratio of the niacinamide and the citric acid is 8:1 to 1:4.5.

The present invention also provides the use of a cosmetic composition for use in the manufacture of cosmetics for reducing the amount of residual niacinamide on the skin, the cosmetic composition including: niacinamide; and citric acid,

wherein the content of the niacinamide is 5% by weight or more based on the total weight of the cosmetic composition, and

the molar ratio of the niacinamide and the citric acid is 8:1 to 1:4.5.

### Advantageous Effects

The cosmetic composition according to the present invention contains both niacinamide and citric acid, so that when the cosmetic composition is applied to the skin, white residual niacinamide does not remain even when the solvent and agent evaporate and/or are absorbed. Through this, usability problems caused by foreign body sensations or white stains caused by residual niacinamide can be addressed.

### BEST MODE

The present invention relates to a cosmetic composition including niacinamide; and citric acid.

Hereinafter, the configuration of the present invention will be described in detail.

When a part is said to “include” a component, this means that other components may be further included, not excluded, unless specifically stated otherwise.

In the present invention, niacinamide is a type of niacin, a member of the vitamin B group, and may be included in a cosmetic composition for skin improvement effects such as brightening, moisturizing, anti-inflammatory and improvement of inflammatory acne, and improvement of wrinkles and elasticity.

Niacinamide is present as a solid at room temperature, and when a cosmetic composition containing a high content of niacinamide is applied to the skin, the solvent and agent are evaporated or absorbed, causing niacinamide to remain white. In the present invention, when niacinamide is used together with citric acid, which will be described below, skin absorption may be improved due to the interaction between niacinamide and citric acid. Through this, when applying the cosmetic composition to the skin, niacinamide does not remain on the skin even when the solvent and agent are evaporated or absorbed, thereby addressing various problems caused by residual niacinamide.

In other words, it may enable more effective skin absorption and skin penetration of niacinamide.

In addition, the skin permeation amount of niacinamide in the cosmetic composition of the present invention may be 1.5 times or more that of a composition not containing citric acid. The said skin permeation amount may be measured according to the method of Example-Experimental Example 3, and is the amount when applied for the same time, where the application time may be 18 hours.

In one embodiment, the content of niacinamide may be 5% by weight or more, 6% by weight or more, 7% by weight or more, 8% by weight or more, 9% by weight or more, 10% by weight or more, 15% by weight or more, or 20% by weight or more, based on the total weight (100% by weight) of the cosmetic composition, and the upper limit may be 50% by weight or less. Specifically, the content of niacinamide may be 5 to 50% by weight, 5 to 20% by weight, 5 to 15% by weight, 6 to 50% by weight, 6 to 20% by weight, 6 to 15% by weight, 7 to 50% by weight, 7 to 20% by weight, 7 to 15% by weight, 8 to 50% by weight, 8 to 20% by weight, 8 to 15% by weight, 9 to 50% by weight, 9 to 20% by weight, 9 to 15% by weight, 10 to 50% by weight, 10 to 20% by weight, 10 to 15% by weight, 15 to 50% by weight, 15 to 20% by weight, or 20 to 50% by weight. When the content of niacinamide is less than 5% by weight, the desired effect of niacinamide cannot be obtained, and when it exceeds 50% by weight, there is a risk of irritating the skin.

In the present invention, citric acid can be used together with niacinamide to improve skin absorption and skin penetration of the niacinamide.

In one embodiment, citric acid may be citric acid monohydrate, as needed.

In one embodiment, the content of citric acid may be 0.9% by weight or more, 1% by weight or more, 1.5% by weight or more, 2.5% by weight or more, 3.6% by weight or more, 7.5% by weight or more, 10% by weight or more, 15% by weight or more, 20% by weight or more, 25% by weight or more, or 30% by weight or more, based on the total weight (100% by weight) of the cosmetic composition. The upper limit of the content may be 50% by weight or less or 40% by weight or less. Specifically, the content of citric acid may be 0.9 to 2.5% by weight, 2.6 to 3.5% by weight, 3.6 to 7.5% by weight, 7.5 to 15% by weight, 16 to 20% by weight, 21 to 30% by weight, 31 to 35% by weight, 10 to 20% by weight, 25 to 35% by weight, 1 to 40% by weight, 1.5 to 35% by weight, 2 to 30% by weight, or 10 to 25% by weight. When the content is less than 0.9% by weight, the composition does not show the effect of improving skin absorption and skin penetration of niacinamide, and when it exceeds 50% by weight, there is a risk of irritating the skin. When citric acid monohydrate is used as the citric acid, the content may be appropriately adjusted based on citric acid.

In one embodiment, the content of citric acid may be 0.9 to 35% by weight based on 5% by weight of niacinamide, the content of citric acid may be 1.5 to 50% by weight based on 10% by weight of niacinamide, and the content of citric acid may be 3 to 50% by weight based on 20% by weight of niacinamide.

In one embodiment, the molar ratio of niacinamide and citric acid may be 8:1 to 1:4.5, 6:1 to 4:1, 4:1 to 1:4, 2:1 to 1:4, 1:1 to 1.4, 4:1 to 1:2, or 4:1 to 1:1. In addition, the weight ratio of niacinamide and citric acid may be 1:0.2 to 1:7.5 or 1:0.3 to 1:7, specifically, 1:0.3 to 1:0.4, 1:0.5 to 1:0.7, 1:0.75 to 1:2.5, 1:2.6 to 1:3.0, 1:3.1 to 1:4.5, 1:4.6 to 1:5.5, or 1:5.6 to 1:7. When the weight ratio is outside the above range, there is a risk of problems such as white residual niacinamide remaining on the skin.

In one embodiment, the cosmetic composition may further include panthenol in addition to citric acid. The present invention uses citric acid to address the problem of residual niacinamide, but when the citric acid is used in excessive amounts, skin irritation problems may occur due to the low pH of the composition, and formulation instability may also occur. Therefore, in the present invention, by further including panthenol, irritation caused by a decrease in pH due to excessive use of citric acid can be alleviated, and the skin absorption and skin penetration of the niacinamide can be further improved.

The content of the panthenol may be 0.05% by weight or more, 0.66% by weight or more, 2.0% by weight or more, 3.4% by weight or more, 5% by weight or more, 6.2% by weight or more, 9.0% by weight or more, 11% by weight or more, 14.6% by weight or more, 23% by weight or more, or 31% by weight or more, based on the total weight (100% by weight) of the cosmetic composition. The upper limit of the panthenol content may be 65% by weight or less, 48% by weight or less, or 31% by weight or less.

In one embodiment, the content of the panthenol may be appropriately adjusted depending on the content of niacinamide and citric acid, and the panthenol may partially replace the citric acid content. For example, the molar ratio of the niacinamide and the sum of the citric acid and the panthenol may be 8:1 to 1:4.5, 6:1 to 4:1, 4:1 to 1:4, 2:1 to 1:4, 1:1 to 1:4, 4:1 to 1:2, or 4:1 to 1:1.

In one embodiment, the cosmetic composition may further include one or more selected from the group consisting of erythritol, xylitol, and xylityl glucoside. The erythritol, xylitol, and/or xylityl glucoside, like panthenol, may relieve irritation caused by a decrease in pH caused by excessive use of citric acid.

The content of erythritol may be 0.03% by weight or more, 0.06% by weight or more, 0.1% by weight or more, or 0.2% by weight or more, based on the total weight (100% by weight) of the cosmetic composition. The upper limit of the content may be 4% by weight or less, 3% by weight or less, 2% by weight or less, 1% by weight or less, or 0.5% by weight or less.

The content of xylitol may be 0.03% by weight or more, 0.07% by weight or more, 0.1% by weight or more, or 0.3% by weight or more, based on the total weight (100% by weight) of the cosmetic composition. The upper limit of the content may be 5% by weight or less, 3.8% by weight or less, 2.5% by weight or less, 1.3% by weight or less, or 0.7% by weight or less.

The content of xylityl glucoside may be 0.08% by weight or more, 0.1% by weight or more, 0.3% by weight or more, or 0.6% by weight or more, based on the total weight (100% by weight) of the cosmetic composition. The upper limit of the content may be 10.3% by weight or less, 7.8% by weight or less, 5.2% by weight or less, 2.6% by weight or less, or 1.3% by weight or less.

In one embodiment, the acidity of the cosmetic composition according to the present invention may be adjusted to have a value of pH 3.5 to pH 10 to improve discomfort such as skin irritation when applied to the skin.

The cosmetic composition according to the present invention may include oily components in addition to the components described above. The oily components may be included in the composition to help niacinamide be present in the composition in a stable manner.

In one embodiment, the oily component may be one or more of oil and wax. All of the oils and waxes commonly used as components of cosmetics in the art may be applied. For example, the oil may be silicone-based oil, ester-based oil, triglyceride-based oil, hydrocarbon-based oil, or vegetable oil, and one or more of these components may be combined as needed.

The silicone-based oil may be a silicone-based fluid oil or a silicone-based crosspolymer dispersed in oil. For example, as silicone-based fluid oils, cyclopentasiloxane, cyclohexasiloxane, cycloheptasiloxane, cyclomethicone, cyclophenyl methicone, cyclotetrasiloxane, cyclotrisiloxane, dimethicone, capryl dimethicone, caprylyl trimethicone, caprylyl methicone, cetearyl methicone, hexadecyl methicone, hexyl methicone, lauryl methicone, myristyl methicone, phenyl methicone, stearyl methicone, stearyl dimethicone, trifluoropropyl methicone, cetyl dimethicone, diphenyl siloxyphenyl trimethicone, dimethylpolysiloxane, methylphenyl polysiloxane, decamethylcyclopentasiloxane, methyl trimethicone, or phenyl trimethicone may be used. The silicone-based oil components may be used alone or in combination of two or more types of oils. Silicone-based crosspolymers include, but are not limited to, a dimethicone crosspolymer, a dimethicone/vinyl dimethicone crosspolymer, a dimethicone PEG-10/15 crosspolymer, or a PEG-12 dimethicone/PPG-20 crosspolymer.

As the ester-based oils, ascorbyl palmitate, ascorbilinolate, ascorbyl stearate, diisostearyl malate, benzyl benzoate, benzyl laurate, butylene glycol dicaprylate/dicaprate, butylene glycol diisononanoate, butylene glycol laurate, butylene glycol stearate, butyl isostearate, cetearyl isononanoate, cetearyl nonanoate, cetyl caprylate, cetylethylhexanoate, cetyl isononanoate, ethylhexyl caprylate/caprate, ethylhexyl isononanoate, ethylhexyl isostearate, ethylhexyllaurate, hexyllaurate, octyldodecyl isostearate, isopropyl isostearate, isostearyl isononanoate, isostearyl isostearate, isocetylethylhexanoate, neopentyl glycol dicaprate, neopentyl glycol diethyl hexanoate, neopentyl glycol diisononanoate, neopentyl glycol diisostearate, pentaerythrityl stearate, pentaery thrityl tetraethylhexanoate, dipentaery thrityl hexaacid ester, polyglyceryl-2-diisostearate, polyglyceryl-2-sesquiisostearate, polyglyceryl-2-isostearate, polyglyceryl-2-tetraisostearate, polyglyceryl-2-triisostearate, polyglyceryl-3-diisostearate, polyglyceryl-3-isostearate, polyglyceryl-4-disostearate, polyglyceryl-4-isostearate, polyglyceryl-6-disostearate, polyglyceryl-6-sesquiisostearate, or triethylhexanoin may be used.

As the triglyceride-based oils, C8-C12 acid triglycerides, C12-C18 acid triglycerides, caprylic/capric/triglyceride, caprylic/capric/lauric triglyceride, C10-C40 isoalkyl acid triglycerides, C10-C18 triglycerides, glyceryl triacetylhydrostearate, soybean glyceride, tribehenin, tricaprine, triethylhexanoin, triheptanoin, triisostearin, tripalmitin, or tristearin may be used.

As the hydrocarbon-based oils, liquid paraffin (liquid paraffin, mineral oil), paraffin, vaseline, microcrystalline wax, or squalene may be used.

As the vegetable oils, avocado oil, wheat germ oil, rosehip oil, shea butter, almond oil, olive oil, macadamia oil, argan oil, meadowfoam oil, sunflower oil, castor oil, camellia oil, corn oil, safflower oil, soy bean oil, rapeseed oil, macadamia nut oil, jojoba oil, palm oil, palm kernel oil, or coconut oil may be used.

As the wax, all waxes such as hydrocarbon-based waxes, vegetable waxes, or silicone waxes generally used in cosmetics may be used. For example, the wax may include candelilla wax, carnauba wax, rice wax, bees wax, lanolin, ozokerite, ceresin wax, paraffin wax, microcrystalline wax, C30-C45 alkyl dimethylsilyl polypropyl silsesquioxane, ethylene/propylene copolymers or polyethylene wax, but is not limited thereto.

The content of the oily component is not particularly limited, and the components may be included in an appropriate amount in the cosmetic composition.

The cosmetic composition of the present invention may additionally include components that may be used in conventional cosmetics, for example, humectants such as glycerin, butylene glycol, propylene glycol, hexanediol, methyl gluceth-20, diglycerin, and ethylhexylglycerin; sunscreens such as ethylhexyl methoxycinnamate, ethylhexyl salicylate, ethylhexyl triazone, octocrylene, and bis-ethylhexyloxyphenol methoxyphenyl triazine; pH-adjusting agents such as triethanolamine; thickeners such as carbomer, xanthan gum, acrylate/C10-30 alkyl acrylate crosspolymers, and hyaluronic acid; preservatives such as phenoxyethanol, methylparaben, and propylparaben; antioxidants such as BHT, ethyl ascorbyl ether, and ascorbic acid; skin conditioning agents such as beta-glucan; surfactants such as cetearyl glucoside and sorbitan stearate; fragrances or pigments, and there is no limitation on the components.

Each of the above components included in the cosmetic composition according to the present invention may be included in the cosmetic composition of the present invention, preferably within the range not exceeding the maximum amount stipulated in the “Cosmetics Safety and Technical Code” prescribed by the Chinese government.

The cosmetic composition according to the present invention may be prepared in any formulation conventionally prepared in the art. For example, the cosmetic composition may include a toner such as a flexible toner or a nourishing toner, a spray-type toner, a facial lotion, an emulsion such as a body lotion, a cream such as a nourishing cream, a moisturizing cream, an eye cream, a stick, an essence, a cosmetic ointment, a spray, a gel, a pack, a sunscreen, a makeup base, foundation such as a liquid or spray type, a powder, a cleansing lotion, a makeup remover such as cleansing oil, a cleansing agent such as cleansing foam, soap, body wash, etc., but is not limited thereto.

In one embodiment, the formulation of the cosmetic composition may be an aqueous formulation, water-in-oil (W/O) formulation, oil-in-water (O/W) formulation, a solubilized formulation, or an oil formulation.

The cosmetic composition of the present invention may be used according to a conventional method of use, and the number of times of use may vary depending on the skin condition or taste of the user.

The cosmetic composition according to the present invention has the effect of significantly improving residual niacinamide on the skin due to solvent evaporation upon application.

In one embodiment, the change in L value measured with a colorimeter before and after application of the cosmetic composition may be 5 or less. When the change in the L value is 5 or less, it can be determined that there is almost no color difference due to residual niacinamide. This change in L value may be measured and calculated according to the method in [Reference] of the Examples.

In addition, the present invention relates to a method of preparing the above-described cosmetic composition.

The cosmetic composition according to the present invention may be prepared by preparing a mixture of niacinamide and citric acid and then mixing other components used in general cosmetic compositions, and may be also prepared by mixing niacinamide, citric acid, and other components at one time.

In one embodiment, a mixture of niacinamide and citric acid may be prepared by mixing niacinamide, citric acid, and water.

The mixing may be performed at 45 to 90° C., 45 to 80° C., or 60 to 80° C. As a result, niacinamide and citric acid are uniformly mixed in water, thereby achieving a skin improvement effect, low irritation to the skin, and excellent formulation stability.

In one embodiment, when preparing a mixture of niacinamide and citric acid, panthenol may be additionally used to prepare the mixture.

In addition, the present invention provides a method of increasing the skin absorption and/or skin penetration of niacinamide using a cosmetic composition including niacinamide; and citric acid,

wherein the content of the niacinamide is 5% by weight or more based on the total weight of the cosmetic composition, and

the molar ratio of the niacinamide and the citric acid is 8:1 to 1:4.5.

The cosmetic composition according to the present invention may further include panthenol, and the molar ratio of the niacinamide and the sum of the citric acid and the panthenol may be 8:1 to 1:4.5.

In addition, the present invention provides a method of reducing the amount of residual niacinamide on the skin using a cosmetic composition including niacinamide; and citric acid,

wherein the content of the niacinamide is 5% by weight or more based on the total weight of the cosmetic composition, and

the molar ratio of the niacinamide and the citric acid is 8:1 to 1:4.5.

The cosmetic composition according to the present invention may further include panthenol, and the molar ratio of the niacinamide and the sum of the citric acid and the panthenol may be 8:1 to 1:4.5.

In addition, the present invention provides the use of a cosmetic composition for use in the manufacture of cosmetics for increasing the skin absorption and/or skin penetration of niacinamide, the cosmetic composition including niacinamide; and citric acid,

wherein the content of the niacinamide is 5% by weight or more based on the total weight of the cosmetic composition, and

the molar ratio of the niacinamide and the citric acid is 8:1 to 1:4.5.

The cosmetic composition according to the present invention may further include panthenol, and the molar ratio of the niacinamide and the sum of the citric acid and the panthenol may be 8:1 to 1:4.5.

The present invention also provides the use of the cosmetic composition for use in the manufacture of cosmetics for reducing the amount of residual niacinamide on the skin, the cosmetic composition including niacinamide; and citric acid,

wherein the content of the niacinamide is 5% by weight or more based on the total weight of the cosmetic composition, and

the molar ratio of the niacinamide and the citric acid is 8:1 to 1:4.5.

The cosmetic composition according to the present invention may further include panthenol, and the molar ratio of the niacinamide and the sum of the citric acid and the panthenol may be 8:1 to 1:4.5.

Hereinafter, the present invention will be described in detail through Examples. The following examples merely illustrate the present invention, but the scope of the present invention is not limited by the following examples. The examples are merely provided to complete the disclosure of the present invention and to fully inform those skilled in the art of the scope of the invention, and the invention is only defined by the scope of the claims.

### MODES OF THE INVENTION

### Examples

### [Reference] Evaluation Method 1: Evaluation of Residual Niacinamide by Naked Eye

Before evaluation, the test subject's forearm skin was washed with water, wiped with a tissue, and left at room temperature for 10 min. Afterward, the sample composition was rubbed and spread onto the test subject's forearm skin and then left at room temperature for 10 min. After 10 min, residues were checked with the naked eye and evaluated as follows.
0: Visible white residue
X: No visible white residue.

After the evaluation, the skin to which the sample composition had been applied was washed with water and wiped with a tissue, and there was a period of 1 hour until the next evaluation.

Evaluation method 2: Evaluation of Residual Niacinamide using Colorimeter

Before evaluation, the test subject's forearm skin was washed with water, wiped with a tissue, and left at room temperature for 10 min. Afterward, marks were made with a black name pen in four directions centered on the area (i.e., forearm skin) where the sample composition was to be applied, and the initial value Lo was measured by measuring the center portion using a colorimeter (chromameter).

The sample composition was rubbed and spread onto the test subject's forearm skin, and left at room temperature for 10 min, and the center portion was measured again using a colorimeter to obtain the measured value Lₑ. The difference between the measured value Lₑ and the initial value L₀ was calculated, and the evaluation value ΔL was recorded.

After the evaluation, the skin to which the sample composition had been applied was washed with water and wiped with a tissue, and there was a period of 1 hour until the next evaluation.

### Experimental Example 1. Evaluation of Effect of Improving Skin Absorption of Niacinamide by Citric Acid

A composition containing niacinamide and citric acid was prepared with the composition and content (% by weight) shown in Table 1 below.

Specifically, citric acid was contained in various molar ratios relative to 5% by weight of niacinamide, and water was used as the remainder to prepare a solution with a total of 100% by weight. The solution was dissolved by stirring at 80° C. for 10 min, and then cooled to room temperature to prepare a composition containing niacinamide and citric acid.

Next, the residual niacinamide was evaluated according to Evaluation method 1 and Evaluation method 2 described above. The results are shown in Table 1.

TABLE 1

| Composition/ | Niacinamide:citric acid | | | | | | | | | |
|---|---|---|---|---|---|---|---|---|---|---|
| molar ratio | 10:0 | 9:1 | 4:1 | 3:1 | 2:1 | 1:1 | 1:2 | 1:3 | 1:4 | 1:5 |
| Niacinamide | 5 | 5 | 5 | 5 | 5 | 5 | 5 | 5 | 5 | 5 |
| Citric acid | — | 0.874 | 1.96 | 2.62 | 3.93 | 7.86 | 15.7 | 23.5 | 31.4 | 39.3 |
| Water | to100 | to100 | to100 | to100 | to100 | to100 | to100 | to100 | to100 | to100 |
| Evaluation | ◯ | ◯ | X | X | X | X | X | X | X | ◯ |
| method 1 | | | | | | | | | | |
| Evaluation | 9.40 | 8.51 | 0.32 | 0.30 | 0.34 | 0.23 | 0.46 | 0.14 | 0.44 | 5.55 |
| method 2 | | | | | | | | | | |

As shown in Table 1, when the molar ratio of niacinamide and citric acid is 4:1 to 1:4, niacinamide does not remain on the skin, and it can be confirmed that the color difference in the skin before and after application of the composition is small.

On the other hand, when citric acid is not contained, or when the content thereof is insufficient or excessive, niacinamide remains, and as a result, it can be confirmed that the color difference of the skin before and after applying the composition is greater than 5.

### Experimental Example 2. Effect of Improving Skin Absorption of Cosmetic Composition Containing Niacinamide and Citric Acid

An aqueous cosmetic composition and an O/W cosmetic composition were prepared with the composition and content (% by weight) shown in Tables 2 and 3 below.

Thereafter, the residual niacinamide was evaluated according to Evaluation method 1 and Evaluation method 2 described above. The results are shown in Tables 2 and 3.

TABLE 2

| | Comparative | Comparative | Example | Example |
|---|---|---|---|---|
| | Example 1 | Example 2 | 1 | 2 |
| Water | to 100 | to 100 | to 100 | to 100 |
| Xanthan gum | 0.30 | 0.30 | 0.30 | 0.30 |
| Trisodium EDTA | 0.02 | 0.02 | 0.02 | 0.02 |
| 1,2-Hexanediol | 1.20 | 1.20 | 1.20 | 1.20 |
| Diethoxyethyl | 3.00 | 3.00 | 3.00 | 3.00 |
| succinate | | | | |
| Methyl gluceth-20 | 1.00 | 1.00 | 1.00 | 1.00 |
| Glycerin | 1.00 | 1.00 | 1.00 | 1.00 |
| Alanyl glutamine | 0.50 | 0.50 | 0.50 | 0.50 |
| Niacinamide | 10.00 | 10.00 | 10.00 | 10.00 |
| Citric acid | 0.00 | 1.74 | 5.00 | 2.00 |
| Panthenol | — | — | — | 5.56 |
| Evaluation method 1 | ◯ | ◯ | X | X |
| Evaluation method 2 | 8.40 | 6.94 | 0.04 | 0.03 |

TABLE 3

| | Comparative | Comparative | Example |
|---|---|---|---|
| | Example 3 | Example 4 | 3 |
| Water | to 100 | to 100 | to 100 |
| Xanthan gum | 0.30 | 0.30 | 0.30 |
| Hydroxyethyl acrylate/sodium | 0.20 | 0.20 | 0.20 |
| acryloyl dimethyl taurate | | | |
| copolymer | | | |
| Trisodium EDTA | 0.02 | 0.02 | 0.02 |
| 1,2-Hexanediol | 1.20 | 1.20 | 1.20 |
| Glycerin | 2.00 | 2.00 | 2.00 |
| Alanyl glutamine | 0.50 | 0.50 | 0.50 |
| Niacinamide | 10.00 | 10.00 | 10.00 |
| Citric acid | 0.00 | 1.74 | 5.00 |
| Cetearyl alcohol | 0.50 | 0.50 | 0.50 |
| Caprylic/capric triglyceride | 3.00 | 3.00 | 3.00 |
| Triethylhexanoin | 2.00 | 2.00 | 2.00 |
| Polysorbate 60 | 0.60 | 0.60 | 0.60 |
| Evaluation method 1 | ◯ | ◯ | X |
| Evaluation method 2 | 6.31 | 5.50 | 0.60 |

As shown in Tables 2 and 3, although the cosmetic compositions prepared in Examples 1 to 3 contain a high content of niacinamide, it can be confirmed that the problem of residual niacinamide can be improved by including citric acid. Particularly, it can be confirmed that Example 2, which contains both citric acid and panthenol, shows an excellent skin absorption effect even at a low citric acid content.

On the other hand, it can be confirmed that in Comparative Examples 1 and 3, which do not contain citric acid, and Comparative Examples 2 and 4, which do not contain sufficient citric acid, niacinamide is not sufficiently absorbed into the skin and remains as a white residue.

### Experimental Example 3. Effect of Improving Skin Penetration of Niacinamide by Citric Acid

The skin penetration improvement effect of the cosmetic composition containing niacinamide and citric acid was confirmed.

A cosmetic composition was prepared with the composition and content (% by weight) shown in Tables 4 and 5 below.

TABLE 4

| | Comparative | Example | Example | Example | Example |
|---|---|---|---|---|---|
| | Example 5 | 4 | 5 | 6 | 7 |
| Niacin- | 20 | 10 | 10 | 20 | 20 |
| amide | | | | | |
| Citric acid | — | 15.7 | 2 | 31.4 | 4 |
| Panthenol | — | — | 5.56 | — | 11.1 |
| Water | to 100 | to 100 | to 100 | to 100 | to 100 |

TABLE 5

| | Comparative | Example |
|---|---|---|
| | Example 6 | 8 |
| Water | To 100 | To 100 |
| Xanthan gum | 0.30 | 0.30 |
| TrisodiumEDTA | 0.02 | 0.02 |
| 1,2-Hexanediol | 1.20 | 1.20 |
| Diethoxyethyl succinate | 3.00 | 3.00 |
| Methyl gluceth-20 | 1.00 | 1.00 |
| Glycerin | 1.00 | 1.00 |
| Alanyl Glutamine | 0.50 | 0.50 |
| Niacinamide | 10.00 | 10.00 |
| Citric acid | 0 | 15.7 |

Afterward, a transdermal absorption experiment was performed using porcine skin.

Specifically, the receptor of a Franz diffusion cell was filled with phosphate-buffered saline (pH 7.4, Gibco), and porcine skin was mounted between the donor and the receptor so that the epidermis faced upward (toward the donor) and the dermis faced downward (toward the receptor). 20 μL of the compositions of Comparative Examples and Examples were applied to the porcine skin, spread evenly, and placed in a thermohygrostat at a temperature of 37° C. and 50% humidity for 18 hours.

Afterward, the same criteria as the above-described Evaluation method 1 were applied to visually determine whether the composition remained on the porcine skin, and the unabsorbed solution was removed and the skin tissue was recovered. The amount of niacinamide in the skin tissue and receptor was quantified using only the skin tissue exposed to the composition among the porcine skin, and the sum of the values was taken as the permeation amount and is shown in Tables 6 and 7.

TABLE 6

| | Comparative | Example | Example | Example | Example |
|---|---|---|---|---|---|
| | Example 5 | 4 | 5 | 6 | 7 |
| Niacin- | 20 | 10 | 10 | 20 | 20 |
| amide (%) | | | | | |
| Residue | ◯ | X | X | X | X |
| on skin | | | | | |
| Perme- | 395 | 555 | 662 | 831 | 953 |
| ation | | | | | |
| amount | | | | | |
| (μg) | | | | | |

TABLE 7

| | Comparative | Example |
|---|---|---|
| | Example 6 | 8 |
| Niacinamide (%) | 10 | 10 |
| Residue on skin | ◯ | X |
| Permeation amount (μg) | 264 | 555 |

First, as shown in Table 6, compared to Comparative Example 5 using 20% niacinamide, it can be confirmed that Examples 4 and 5 exhibit a larger amount of skin penetration even though the content of niacinamide is only 10%.

In addition, as shown in Table 7, it can be confirmed that Example 8, which contains citric acid in a cosmetic composition containing a high amount of niacinamide as in the present invention, has a significantly higher skin permeation amount of niacinamide than Comparative Example 6, which does not contain citric acid.

Through this, it can be confirmed that the niacinamide of the Examples can more easily penetrate the lipid layer of the stratum corneum and be delivered to the skin in greater amounts, and that skin absorption is improved.

### Industrial Applicability

The cosmetic composition according to the present invention contains both niacinamide and citric acid, so that when the cosmetic composition is applied to the skin, residual white niacinamide does not remain even when the solvent and agent evaporate and/or are absorbed. Through this, usability problems caused by foreign body sensation or white stains caused by residual niacinamide can be addressed.

1. A cosmetic composition comprising niacinamide and citric acid,
wherein the niacinamide is in an amount of 5% by weight or more based on the total weight of the cosmetic composition, and
the niacinamide and the citric acid is in a molar ratio of 8:1 to 1:4.5.

2. The cosmetic composition of claim 1, wherein the skin absorption and/or skin penetration of the niacinamide are/is increased.

3. The cosmetic composition of claim 1, wherein a skin permeation amount of the niacinamide in the cosmetic composition is 1.5 times or more that of a composition not containing the citric acid.

4. The cosmetic composition of claim 1, wherein the citric acid is in an amount of 0.9 to 50% by weight based on the total weight of the cosmetic composition.

5. The cosmetic composition of claim 1, further comprising panthenol.

6. The cosmetic composition of claim 5, wherein the panthenol is in an amount of 0.05% by weight or more based on the total weight of the cosmetic composition.

7. The cosmetic composition of claim 5, wherein a molar ratio of the niacinamide and the sum of the citric acid and the panthenol is 8:1 to 1:4.5.

8. The cosmetic composition of claim 1, wherein the cosmetic composition has a pH of 3.5 to 10.

9. A method of increasing skin absorption and/or skin penetration of niacinamide when administered to the skin of a subject in need thereof, comprising applying the cosmetic composition of claim 1 to the skin of the subject.

10. A method of reducing residual niacinamide on the skin when administered to the skin of a subject in need thereof, comprising applying the cosmetic composition of claim 1 to the skin of the subject.
